(19) United States
(12) Patent Application Publication
(10) Pub. No.: US 2025/0384558 A1
(43) Pub. Date: Dec. 18, 2025

(54) PROVIDING A CLASSIFIED DATA SET

(71) Applicant: Siemens Healthineers AG, Forchheim (DE)

(72) Inventors: Annette Birkhold, Stuttgart (DE); Philipp Roser, Erlangen (DE); Markus Kowarschik, Erlangen (DE)

(21) Appl. No.: 19/221,304

(22) Filed: May 28, 2025

(30) Foreign Application Priority Data
Jun. 13, 2024 (DE) 10 2024 205 456.9
Jun. 14, 2024 (DE) 10 2024 205 509.3

(51) Int. Cl. G06T 7/00; A61B 6/00; G06T 7/215; G06T 11/00; G06V 10/26; G06V 10/764; G06V 10/82; G06V 40/10; G16H 30/40

(52) U.S. Cl. CPC G06T 7/0016; A61B 6/5288; G06T 7/215; G06T 11/008; G06V 10/764; G16H 30/40; A61B 6/481; G06T 2207/10081; G06T 2207/10116; G06T 2207/20081; G06T 2207/20084; G06T 2207/30008; G06T 2207/30104; G06T 2207/30196; G06T 2211/404; G06V 10/273; G06V 10/82; G06V 40/10; G06V 2201/033

(57) ABSTRACT

A method for providing a classified data set includes capturing an image data set of an examination object by a medical imaging device. The image data set has a plurality of image points in each case with a time-intensity curve. The image points map an examination area of the examination object with at least one contrast-enhanced vascular section. The method further includes identifying first image points in the image data set whose time-intensity curves have a predefined variability as image points that map the at least one contrast-enhanced vascular section, and providing the classified data set based on the image data set and the first image points, wherein the classified data set has a classification between the first image points and further image points of the image data set.

The present patent document claims the benefit of German Patent Application No. 10 2024 205 456.9, filed Jun. 13, 2024, and German Patent Application No. 10 2014 205 509.3, filed Jun. 14, 2024, which are hereby incorporated by reference in their entireties.

### TECHNICAL FIELD

The present disclosure relates to a method for providing a classified data set, a medical imaging device, and a computer program product.

### BACKGROUND

X-ray imaging is frequently used to capture temporal changes in an examination area of an examination object. The temporal and/or spatial change to be captured may include a spreading motion or and/or flow motion of a contrast agent, in particular a contrast agent flow and/or a contrast agent bolus, in a hollow organ, (e.g., in a vascular section), of the examination object.

Herein, X-ray imaging methods may include digital subtraction angiography (DSA), wherein at least two chronologically recorded X-ray images that at least partially map the common examination area are subtracted from one another. In addition, in DSA, a distinction is frequently made between a mask phase for recording at least one mask image and a contrast-enhanced phase for recording at least one contrast-enhanced image. Herein, the mask image may map the examination area without contrast agent, in particular without contrast agent within the examination area. Furthermore, the contrast-enhanced image may map the examination area with contrast agent, in particular when the contrast agent is within the examination area. A differential image is frequently provided as the result of DSA by subtracting the mask and contrast-enhanced image from each other. In this way, the components in the differential image that are irrelevant and/or disruptive to treatment and/or diagnosis, which in particular do not change over time, may be reduced and/or removed.

Three-dimensional (3D) spatially resolved DSA (3D-DSA) is a commonly performed act in the assessment of cerebral vessels to prevent bone structures interfering with visualization. Herein, 3D-DSA is based on an initial mask run, in particular the recording of the mask image, without contrast agent injection. This is suboptimal from both from a clinical point of view and in terms of usability. Motions of the examination object between recordings may give rise to unwanted artifacts. Furthermore, the contrast agent has to be injected at an exact time. Furthermore, recording takes twice as long due to the mask and contrast-enhanced phases. In addition, an additional X-ray dose is applied during the mask run.

### SUMMARY AND DESCRIPTION

It is therefore the object of the present disclosure to enable contrast-enhanced vascular sections to be captured efficiently in terms of time and X-ray dose.

The scope of the present disclosure is defined solely by the appended claims and is not affected to any degree by the statements within this summary. The present embodiments may obviate one or more of the drawbacks or limitations in the related art.

In a first aspect, the disclosure relates to a method, in particular a computer-implemented method, for providing a classified data set. In a first act, an image data set of an examination object is captured by a medical imaging device. Herein, the image data set has a plurality of image points in each case with a time-intensity curve. In addition, the image points form an examination area of the examination object with at least one contrast-enhanced vascular section, in particular in a time-resolved manner. In a further act, first image points in the image data set whose time-intensity curves have a predefined variability are identified as image points that map the at least one contrast-enhanced vascular section. In a further act, the classified data set is provided based on the image data set and the first image points. Herein, the classified data set has a classification between the first image points and further image points of the image data set.

The examination object may be a human and/or veterinary patient and/or an examination phantom, in particular a vascular phantom. The examination object may have a vascular section, in particular an artery or vein.

Capturing the image data set may include capturing and/or reading a computer-readable data memory and/or receiving the image data set from a data memory unit, for example, a database. Furthermore, the image data set may be provided by a processing unit of a medical imaging device for recording the image data set. Advantageously, the contrast agent is located in the at least one vascular section during recording of the image data set. Advantageously, during recording of the image data set, the vascular section is contrast-enhanced, in particular completely and/or uniformly, in particular homogeneously and/or consistently contrast-enhanced. The medical imaging device may include a magnetic resonance imaging (MRI) system, a computed tomography (CT) system, a medical X-ray device, (e.g., a medical C-arm X-ray device), an ultrasound device, a positron emission tomography (PET) system, or a combination thereof.

The image data set may include two-dimensional (2D) and/or three-dimensional (3D) spatially resolved mapping of the examination area of the examination object, in particular the at least one contrast-enhanced vascular section. In addition, the image data set may be time-resolved. Advantageously, the image data set may map a contrast agent, in particular a radiopaque contrast agent, in the at least one vascular section as the at least one contrast-enhanced vascular section. The image data set may have a plurality of image points, in particular pixels and/or voxels, in each case with a time-intensity curve. The time-intensity curves may map the temporal course of image values, in particular intensity values and/or attenuation values, of the respective image points. The examination area may include a spatial area, (e.g., a volume), of the examination object, in particular a region of interest, which includes the at least one contrast-enhanced vascular section.

The identification of the first image points in the image data set whose time-intensity curves have a predefined variability, (e.g., a predefined frequency and/or a predefined frequency component), may include segmenting the first image points. In particular, the identification of the first image points may include a variability analysis, in particular a frequency analysis, of the respective time-intensity curves. Herein, in each case at least one variability, in particular in each case at least one frequency of the variability and/or at least one frequency value of the variability, in particular the temporal change of the image values of the image points, may be identified. In particular, in each case a plurality of variabilities, in particular in each case a plurality of frequencies and/or frequency values, of the image values of the image points, in particular the temporal change of the image values of the image points, may be identified. Furthermore, the respective at least one identified variability, in particular the respective at least one frequency and/or the respective at least one frequency value, may be compared with the predefined variability, in particular a predefined frequency and/or a predefined frequency value. In particular, the respective plurality of variabilities, in particular the respective plurality of frequencies and/or frequency values, may be compared with the predefined variability, in particular the predefined frequency and/or the predefined frequency value. If the respective at least one variability, in particular the respective at least one frequency and/or the respective at least one frequency value, matches the predefined variability, in particular the predefined frequency and/or the predefined frequency value, the respective image point may be identified as a first image point, in particular as the image point that maps the at least one contrast-enhanced vascular section. In particular, the respective image point may be identified as a first image point, in particular as an image point that maps the at least one contrast-enhanced vascular section, if in each case at least one variability of the plurality of variabilities, in particular in each case at least one frequency of the plurality of frequencies and/or in each case at least one frequency value of the plurality of frequency values, matches the predefined variability, in particular the predefined frequency and/or the predefined frequency value. The time-intensity curves of image points of the image data set which map the at least one contrast-enhanced vascular section may advantageously map a motion, in particular a non-rigid, pulsatile and/or cyclic motion, of the at least one contrast-enhanced vascular section. The time-intensity curves of the image points of the image data set which map the at least one contrast-enhanced vascular section may map additional motions, in particular physiological motions, of the examination object, for example, a respiratory motion. Hence, the time-intensity curves of the image points of the image data set may map a superposition of a plurality of motions, in particular physiological motions, of the examination object. Furthermore, the time-intensity curves of the image points of the image data set may exhibit a superposition of a plurality of frequencies corresponding to the motions of the examination object mapped in each case. In contrast, time-intensity curves of the further image points, in particular image points that do not map the at least one vascular section, may map at least one motion, in particular a physiological motion, of the tissue of the examination object that is mapped in each case that is different from the pulsatile motion, for example, a respiratory motion.

The predefined variability, in particular the predefined frequency, may be predetermined or ascertained. According to a first variant, the predefined variability may be predetermined on the basis of user input. Alternatively, or additionally, the predefined variability may be determined on the basis of a physiological parameter, in particular a physiological parameter of the examination object. The physiological parameter may include a heart rate, in particular a pulse rate, of the examination object. The predefined variability may be characterized by one or more predefined frequencies and/or a predefined amplitude.

The provision of the classified data set may include storing the classified data set on a computer-readable storage medium and/or displaying the classified data set on a representation unit and/or transferring the classified data set to a processing unit. In particular, a graphical representation of the classified data set may be displayed by the representation unit.

Advantageously, the classified data set may be provided based on the first image points and the further image points of the image data set. Herein, the classified data set may have a classification, (e.g., annotation and/or masking), between the first image points and the further image points, in particular remaining image points, of the image data set.

The proposed method may advantageously enable classification of the image points of the image data set, in particular at least the first and further image points, without a mask run. Herein, the classification of the image points may advantageously be based on an analysis of a temporal pattern of the time-intensity curves of the image points. This may advantageously enable contrast-enhanced vascular sections to be captured efficiently in terms of time and X-ray dose.

In a further advantageous embodiment of the proposed method, the predefined variability may include a heart rate of the examination object.

Advantageously, the predefined variability may be predetermined in such a way that it includes, in particular at least partially, the heart rate of the examination object. Herein, the predefined variability may be based on a statistical heart rate of a plurality of examination objects or the, in particular instantaneous or average, heart rate of the examination object.

The proposed embodiment may advantageously enable improved identification, in particular classification, of image points of the image data set that map at least one, in particular arterial and/or venous, blood vessel section.

In a further advantageous embodiment of the proposed method, second image points whose time-intensity curves are constant may be identified as image points in the image data set that map at least one bone tissue. Herein, the classified data set may additionally be provided based on the second image points. Furthermore, the classified data set may have a classification at least between the first and the second image points.

The second image points may be identified in an analogous manner to the identification of the first image points. Herein, the identification of the second image points may include segmenting the second image points. In particular, the identification of the second image points may include a variability analysis, in particular a frequency analysis, of the respective time-intensity curves. Herein, the respective at least one variability, in particular the respective at least one frequency and/or the respective at least one frequency value, of the time-intensity curve of the respective image point may be compared with a variability threshold value, in particular a frequency threshold value. If the respective at least one variability, in particular the respective at least one frequency and/or the respective maximum frequency value, falls below the variability threshold value, the respective image point may be identified as a second image point, in particular as an image point that maps bone tissue.

Advantageously, the classified data set may additionally be provided based on the second image points of the image data set, in particular based on the first image points, the second image points and the further image points of the image data set.

The classified data set may have a classification, (e.g., annotation and/or masking), at least between the first image points and the second image points of the image data set. Advantageously, the classified data set may further have a classification between the first image points, the second image points and the further image points, in particular the remaining, image points, of the image data set.

The proposed embodiment may advantageously enable classification, in particular differentiation, that is efficient in terms of time and X-ray dose between image points of the image data set which map the at least one vascular section or bone tissue.

In a further advantageous embodiment of the proposed method, the provision of the classified data set may include providing a graphical representation depending on the classification of the image points. Herein, the graphical representation may include at least the first image points.

Advantageously, the provision of the graphical representation may include displaying the graphical representation by a representation unit. Herein, the graphical representation may be provided depending on the classification of the image points, in particular the first and further image points. For example, the graphical representation may have color coding and/or annotation and/or masking depending on the classification of the image points. Herein, the graphical representation may advantageously have the first image points, in particular the image values and/or time-intensity curves of the first image points. During the provision of the graphical representation, the further image points may be depicted as masked and/or at least partially transparent. In particular, in the case of 3D spatially resolved mapping of the examination area in the image points, the graphical representation may include a virtual 2D projection of the image points onto a virtual representation plane.

The proposed embodiment may advantageously provide assistance to a medical operator that is efficient in terms of time and X-ray dose during the capture of contrast-enhanced vascular sections.

In a further advantageous embodiment of the proposed method, the second image points may be excluded from the graphical representation.

Advantageously, the second image points may be excluded, in particular masked, from the graphical representation. In particular, in the case of 3D spatially resolved mapping of the examination area in the image points, the graphical representation may include a virtual 2D projection of the image points onto a virtual representation plane. Herein, the second image points may be represented as at least partially transparent, in particular completely transparent.

The proposed embodiment may advantageously provide assistance to a medical operator that is efficient in terms of time and X-ray dose during the capture of contrast-enhanced vascular sections, in particular without obscuring and/or superposition by the image points that map the bone tissue.

In a further advantageous embodiment of the proposed method, the medical imaging device may be embodied as a medical X-ray device. Herein, the capture of the image data set may include capturing projection images of the examination object, in particular the at least one contrast-enhanced vascular section, by the X-ray device.

Advantageously, the X-ray device may include an X-ray source and an X-ray detector, in particular a flat-panel detector and/or a line detector. The X-ray source and the X-ray detector may be arranged in a defined arrangement relative to one another, in particular opposite to one another. Furthermore, the defined arrangement of X-ray source and X-ray detector may be mounted movably, in particular rotatably and/or translatably, for example, with respect to the examination object. The X-ray source may be embodied to emit X-rays for transillumination of the examination object. In particular, the X-ray source may be embodied to emit an X-ray cone beam or an X-ray fan beam for transillumination of the examination object. Herein, a central beam and/or middle beam of the X-rays emitted by the X-ray source may define a projection direction. The X-ray detector may be embodied to detect the X-rays, in particular after interaction with the examination area of the examination object. Furthermore, the X-ray detector may be embodied to provide the projection images depending on the detected X-rays.

The X-ray device may be embodied as a C-arm X-ray device, an O-arm X-ray device, or a computed tomography system (CT system).

The proposed embodiment may advantageously enable the contrast-enhanced vascular sections to be captured efficiently in terms of time and X-ray dose by the X-ray device.

In a further advantageous embodiment of the proposed method, a motion field may be identified, in particular reconstructed, based on the projection images. Herein, the first image points may additionally be identified based on the motion field.

The motion field may have a spatially and temporally resolved representation, in particular 2D or 3D, in particular a model, of the motion of the at least one contrast-enhanced vascular section mapped in the projection images, in particular a motion of the contrast agent in the at least one vascular section. The motion field may be reconstructed based on the projection images. In particular, the motion field may be reconstructed based on the projection images analogously to a 4D reconstruction. Furthermore, the motion field may represent, in particular model, a change in the positioning of the at least one contrast-enhanced vascular section in a spatially and temporally resolved manner. The motion field may have a vector or tensor field representing the motion of the at least one contrast-enhanced vascular section. Herein, the motion represented, in particular modeled, in the motion field of the at least one contrast-enhanced vascular section may include a translation and/or rotation and/or deformation of the at least one contrast-enhanced vascular section. Advantageously, the first image points may additionally be identified based on the motion field. Herein, the motion field may advantageously be determined based on the time-intensity curves of the image points of the image data set.

In particular, the motion field may be used to identify a variability, in particular an image point-by-image point variability, in particular frequency, of the modeled motion. The identification of the first image points may include a comparison of the identified variability, in particular frequency, with the predefined variability, in particular the predefined frequency.

The proposed embodiment may advantageously enable contrast-enhanced vascular sections to be captured reliably based on a motion mapped in the image data set.

In a further advantageous embodiment of the proposed method, the projection images may map the examination object, in particular the at least one contrast-enhanced vascular section, from at least partially different projection directions. Herein, the image data set may be reconstructed from the plurality of projection images.

Advantageously, the X-ray device may capture a plurality of projection images which map the examination object from at least partially different, in particular completely different, projection directions and jointly map at least the examination area. In particular, the plurality of projection images may be captured around a common isocenter. Advantageously, the defined arrangement of X-ray source and X-ray detector may be positioned, in particular moved, along a defined recording trajectory for capturing, in particular recording, the plurality of projection images.

Advantageously, the image data set may be reconstructed from the plurality of projection images, for example, by back projection, in particular filtered back projection. In particular, the reconstruction may take place analogously to the reconstruction of 4D DSA. Herein, the image data set may advantageously map the examination area in 3D with spatial and temporal resolution.

The proposed embodiment may advantageously enable contrast-enhanced vascular sections to be captured in 3D efficiently in terms of time and X-ray dose.

In a further advantageous embodiment of the proposed method, a constraining area that maps a common examination area of the examination object may be identified in the projection images. Herein, the reconstruction of the image data set may be restricted to the constraining area.

The constraining area may include a spatial constraining volume and/or a spatial constraining surface, in particular at least partially within the examination object. Advantageously, the constraining area may be identified as the spatial area of the examination object that is mapped by each projection image of the plurality of projection images, for example, an overlap area. The identification of the constraining area may include segmenting image points of the projection images, (e.g., based on a threshold comparison or a global threshold comparison, in particular a comparison of image values of image points of the projection images with a predetermined threshold value, and/or based on a vesselness filter). Advantageously, the constraining area includes mapping the at least one vascular section, in particular additionally mapping bone tissue, of the examination object.

Advantageously, the reconstruction of the image data set may be restricted to the constraining area, in particular the respective common spatial area in the projection images.

The proposed embodiment may advantageously enable improved, in particular robust and/or low-artifact, reconstruction of the image data set from the projection images.

In a further advantageous embodiment of the proposed method, the identification of the first image points may be based on machine learning.

Advantageously, the identification of the first image points may be based on machine learning, in particular the application of a trained function to the image data set, in particular the time-intensity curves of the image points of the image data set, as input data. Herein, input data of the trained function may be based on the image data set, in particular include the image data set. Furthermore, the classified data set, in particular an identification of the first image points, may be provided as output data of the trained function.

The trained function may be trained by a machine learning method. In particular, the trained function may be a neural network, in particular a convolutional neural network (CNN) or a network including a convolutional layer.

The trained function maps input data to output data. In this case, the output data may furthermore depend on one or more parameters of the trained function. The one or more parameters of the trained function may be determined and/or adapted by training. The determination and/or adaptation of the one or more parameters of the trained function may be based on a pair including training input data and associated training output data, in particular comparison output data, wherein the trained function is applied to provide the classified data set. In particular, the determination and/or adaptation may be based on a comparison of the training mapping data and the training output data, in particular the comparison output data. A trainable function, e.g., a function with one or more parameters that have not yet been adapted, may also be referred to as a trained function.

Other terms for trained functions are trained mapping rule, mapping rule with trained parameters, function with trained parameters, algorithm based on artificial intelligence, machine learning algorithm. An example of a trained function is an artificial neural network, wherein the edge weights of the artificial neural network correspond to the parameters of the trained function. Instead of the term “neural network,” it is also possible to use the term “neural net.” In particular, a trained function may also be a deep neural network or deep artificial neural network. A further example of a trained function is a “support vector machine.” Furthermore, other machine learning algorithms may also be used as trained functions.

Advantageously, the trained function, in particular the neural network, has an input layer and an output layer. Herein, the input layer of the trained function may be embodied to receive the input data. Furthermore, the output layer may be embodied to provide mapping data, in particular output data. Furthermore, the input layer and/or the output layer may in each case include a plurality of channels, in particular neurons.

Advantageously, at least one parameter of the trained function may be adapted based on a comparison of a classified training data set with a comparison data set, for example, a segmented or annotated image data set.

In addition, the trained function may be embodied to identify the second image points in the image data set. Herein, the trained function may advantageously provide the classified data set, in particular an identification of the first and second image points, as output data.

The proposed embodiment may advantageously enable improved, in particular time-efficient, identification of the first image points of the image data set, in particular classification of the image points of the image data set.

In a further advantageous embodiment of the proposed method, the identification of the first image points may be additionally based on the time-intensity curves of image points within a neighboring region of the respective image point.

The neighboring region may include one or more image points of the image data set which adjoin the respective image point, in particular directly. Furthermore, the neighboring region may include a plurality of associated image points of the image data set, which are arranged within a predetermined spatial distance, in particular a radius, with respect to the respective image point, wherein at least one image point of the plurality of image points adjoins the respective image point, in particular directly.

Advantageously, the time-intensity curves of the respective image point and the image points within the neighboring region may be used to determine a motion, for example, an optical flow. Determining the optical flow based on the time-intensity curves of the respective image point and the image points within the neighboring region may include determining motion vectors, in particular a vector field. Including the image points within the neighboring region of the respective image point furthermore enables a motion direction, in particular a flow direction, of the mapped motion to be identified based on the time-intensity curves.

The proposed embodiment may advantageously enable improved identification of the first image points.

In a second aspect, the disclosure relates to a medical imaging device embodied to execute a proposed method for providing a classified data set.

The advantages of the proposed imaging device correspond to the advantages of the proposed method for providing a classified data set. Features, advantages or alternative embodiments mentioned herein may also be transferred to the other claimed subject matter and vice versa.

The medical imaging device may be a magnetic resonance imaging (MRI) system, a computed tomography (CT) system, a medical X-ray device, (e.g., a medical C-arm X-ray device), an ultrasound device, a positron emission tomography (PET) system, or a combination thereof.

Advantageously, the medical imaging device may be embodied to capture the image data set. Furthermore, the imaging device may have a processing unit and an interface. Herein, the processing unit may be embodied to identify the first image points in the image data set. Furthermore, the processing unit and/or interface may be embodied to provide the classified data set.

In a further advantageous embodiment of the proposed imaging device, the imaging device may be embodied as a medical X-ray device. Furthermore, the X-ray device may be embodied to capture projection images of the examination object.

The medical X-ray device may advantageously include a detector, in particular an X-ray detector, and a source, in particular an X-ray source, arranged in a defined arrangement relative to one another, for example, on a C-arm. To record the projection images of the examination object, the X-ray source may emit X-rays to transilluminate the examination object. The detector may be embodied to capture the X-rays after interaction of the X-rays with the examination object. The processing unit may capture the projection images of the examination object on the basis of a signal provided by the detector in dependence on the captured X-rays.

In a third aspect, the disclosure relates to a computer program product with a computer program, which may be loaded directly into a memory of a processing unit, with program sections for processing all acts of a proposed method for providing a classified data set when the program sections are executed by the processing unit.

Herein, the computer program product may include software with a source code that still has to be compiled and linked or only has to be interpreted or an executable software code that only has to be loaded into the processing unit for execution. The computer program product enables the method for providing a classified data set by a processing unit to be executed quickly, identically repeatedly and robustly. The computer program product is configured such that it may execute the method acts by the processing unit.

The computer program product is, for example, stored on a non-transitory computer-readable storage medium or held on a network or server from where it may be loaded into the processor of a processing unit which may be directly connected to the processing unit or be embodied as part of the processing unit. Furthermore, control information of the computer program product may be stored on an electronically readable data carrier. The control information of the electronically readable data carrier may be embodied such that it may perform a method when the data carrier is used in a processing unit. Examples of electronically readable data carriers are DVDs, magnetic tapes or USB sticks on which electronically readable control information, in particular software, is stored. When this control information is read from the data carrier and stored in a processing unit, all embodiments of the above-described methods may be performed.

A software-based implementation has the advantage that it is possible to retrofit processing units used to date in a simple way via a software update in order to work in the manner according to the disclosure. In addition to the computer program, such a computer program product optionally includes additional features, (e.g., documentation), and/or additional components, including hardware components, such as, for example, hardware keys (dongles etc.) for using the software.

### BRIEF DESCRIPTION OF THE DRAWINGS

Exemplary embodiments are shown in the drawings and are described in more detail below. The same reference symbols are used for the same features in the different figures.

FIG. 1 to FIG. 4 depict schematic representations of different embodiments of a proposed method for providing a classified data set.

FIG. 5 depicts a schematic representation of an exemplary time-intensity curve of an image point showing the at least one vascular section.

FIG. 6 depicts a schematic representation of an example of a medical imaging device.

### DETAILED DESCRIPTION

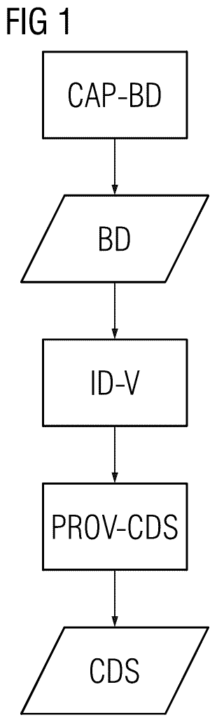

FIG. 1 is a schematic representation of an advantageous embodiment of a proposed method for providing a classified data set. In a first act, an image data set BD of an examination object may be captured CAP-BD by a medical imaging device. Herein, the image data set BD may have a plurality of image points in each case with a time-intensity curve. Furthermore, the image points may map an examination area of the examination object with at least one contrast-enhanced vascular section, in particular in a time-resolved manner. In a further act, first image points in the image data set BD whose time-intensity curves have a predefined variability may be identified ID-V as image points that map the at least one contrast-enhanced vascular section. In a further act, the classified data set CDS may be provided PROV-CDS based on the image data set BD and the first image points. Herein, the classified data set CDS may have a classification between the first image points and further image points of the image data set BD. Advantageously, the predefined variability may include a heart rate of the examination object. Advantageously, the identification ID-V of the first image points may be based on machine learning.

Furthermore, the identification ID-V of the first image points may additionally be based on the time-intensity curves of image points within a neighboring region of the respective image point.

The provision PROV-CDS of the classified data set CDS may include providing a graphical representation depending on the classification of the image points. Herein, the graphical representation may include at least the first image points. In particular, the second image points may be excluded from the graphical representation, in particular masked.

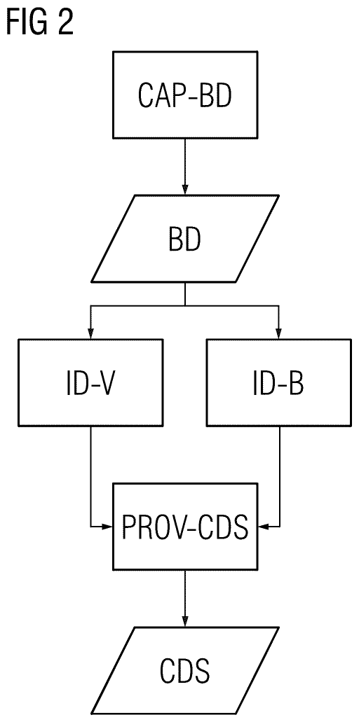

FIG. 2 is a schematic representation of a further advantageous embodiment of a proposed method for providing a classified data set. Herein, second image points whose time-intensity curves are constant may be identified ID-B as image points in the image data set BD that map at least one bone tissue. Furthermore, the classified data set CDS may additionally be provided PROV-CDS based on the second image points. Herein, the classified data set CDS may have a classification at least between the first and the second image points of the image data set BD.

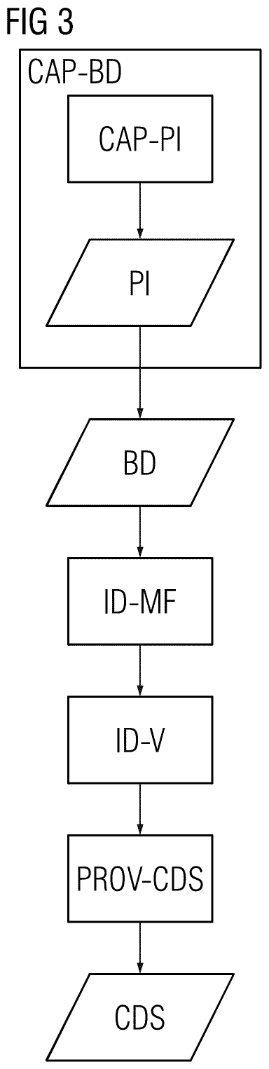

FIG. 3 is a schematic representation of a further advantageous embodiment of a proposed method for providing a classified data set. The medical imaging device may be embodied as a medical X-ray device. Herein, the capturing CAP-BD of the image data set BD may include capturing CAP-PI projection images PI of the examination object by the X-ray device. Furthermore, a 3D motion field may be identified ID-MF based on the projection images PI. Advantageously, the first image points may be additionally identified ID-V based on the 3D-motion field MF.

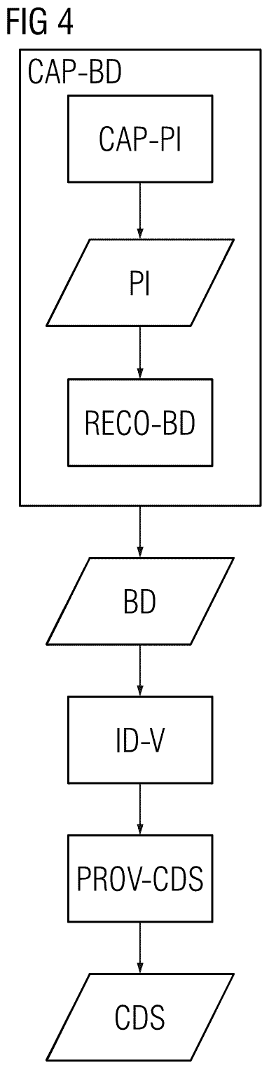

FIG. 4 is a schematic representation of a further advantageous embodiment of a proposed method for providing a classified data set. Herein, the projection images PI may map the examination object from at least partially different projection directions. Furthermore, the image data set BD may be reconstructed RECO-BD from the plurality of projection images PI. Advantageously, a constraining area that maps a common examination area of the examination object may be identified in the projection images PI. Herein, the reconstruction of the image data set RECO-BD may be restricted to the constraining area.

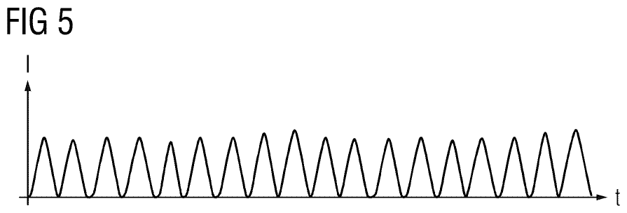

FIG. 5 is a schematic representation of an exemplary time-intensity curve I(t) of an image point showing the at least one vascular section, in particular a first image point. Herein, the time-intensity curve I(t), in particular the temporal change in intensity I mapped by the time-intensity curve I(t), may have a variability, in particular frequency, which includes the predefined variability, in particular a heart rate of the examination object.

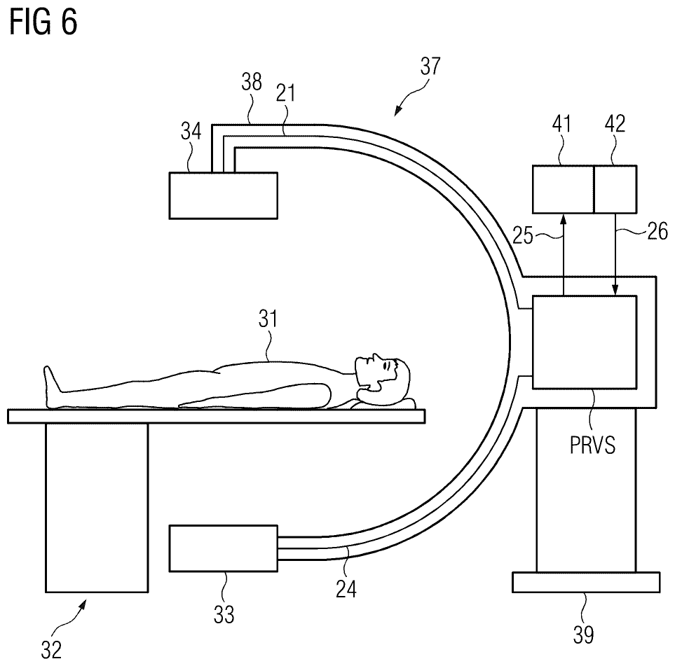

FIG. 6 shows by way of example for a medical imaging device a schematic representation of a medical C-arm X-ray device 37 including a processing unit PRVS. The medical C-arm X-ray device 37 may advantageously have a detector 34, in particular an X-ray detector, and a source 33, in particular an X-ray source, which are arranged in a defined arrangement on a C-arm 38. The C-arm 38 of the C-arm X-ray device 37 may be mounted movably around one or more axes. To record projection images PI of the examination object 31 positioned on a patient support apparatus 32, the processing unit PRVS may send a signal 24 to the X-ray source 33. The X-ray source 33 may then emit an X-ray beam. When the X-ray beam impinges on a surface of the detector 34 after interaction with the examination object 31, the detector 34 may send a signal 21 to the processing unit PRVS. The processing unit PRVS may use the signal 21 to capture CAP-PI the projection images PI and reconstruct RECO-BD the image data set BD. The processing unit PRVS may further be embodied to identify the first image points ID-V in the image data set BD.

The C-arm X-ray device 37 may further have an input unit 42, (e.g., a keyboard), and a representation unit 41, (e.g., a monitor and/or a display and/or a projector). The input unit 42 may preferably be integrated into the representation unit 41, for example, in the case of a capacitive and/or resistive input display. The input 42 may advantageously be embodied to capture user input. For this purpose, the input unit 42 may send a signal 26 to the processing unit PRVS. The processing unit PRVS may be embodied to be controlled in dependence on the user input, in particular the signal 26, in particular for executing a method for providing PROV-CDS a classified data set CDS.

The representation unit 41 may advantageously be embodied to display a graphical representation of the classified data set CDS. For this purpose, the processing unit PRVS may send a signal 25 to the representation unit 41.

The schematic representations contained in the figures described do not represent any scale or proportions.

Finally, it should be noted once again that the methods described in detail above and the apparatuses represented are only exemplary embodiments which may be modified by the person skilled in the art in a wide variety of ways without departing from the scope of the disclosure. Furthermore, the use of the indefinite articles “a” or “an” does not exclude the possibility that the features in question may be present multiple times. Likewise, the terms “unit” and “element” do not exclude the possibility that the components in question include a plurality of interacting subcomponents that may also be spatially distributed if necessary.

In the context of the present application, the expression “based on” may be understood in the sense of the expression “using”. In particular, a formulation according to which a first feature is generated (alternatively: ascertained, determined, etc.) based on a second feature does not exclude the possibility that the first feature may be generated (alternatively: ascertained, determined, etc.) based on a third feature.

1. A method for providing a classified data set, the method comprising:
capturing an image data set of an examination object by a medical imaging device, wherein the image data set has a plurality of image points in each case with a time-intensity curve, and wherein the plurality of image points maps an examination area of the examination object with at least one contrast-enhanced vascular section;
identifying first image points of the plurality of image points in the image data set comprising time-intensity curves that have a predefined variability as image points that map the at least one contrast-enhanced vascular section; and
providing the classified data set based on the image data set and the first image points,
wherein the classified data set has a classification between the first image points and further image points of the image data set.

2. The method of claim 1, wherein the predefined variability comprises a heart rate of the examination object.

3. The method of claim 1, further comprising:
identifying second image points of the plurality of image points in the image data set comprising time-intensity curves that are constant as image points that map at least one bone tissue,
wherein the classified data set is additionally provided based on the second image points, and
wherein the classified data set has a classification at least between the first image points and the second image points of the image data set.

4. The method of claim 3, wherein the providing of the classified data set comprises providing a graphical representation depending on the classification of the image points,
wherein the graphical representation comprises at least the first image points.

5. The method of claim 4, wherein the second image points are excluded from the graphical representation.

6. The method of claim 1, wherein the providing of the classified data set comprises providing a graphical representation depending on the classification of the image points,
wherein the graphical representation comprises at least the first image points.

7. The method of claim 1, wherein the medical imaging device is a medical X-ray device, and
wherein the capturing of the image data set comprises capturing projection images of the examination object by the X-ray device.

8. The method of claim 7, further comprising:
identifying a motion field based on the projection images,
wherein the first image points are additionally identified based on the motion field.

9. The method of claim 7, wherein the projection images map the examination object from at least partially different projection directions, and
wherein the image data set is reconstructed from the plurality of projection images.

10. The method of claim 9, further comprising:
identifying a constraining area in the projection images that maps a common examination area of the examination object,
wherein the reconstruction of the image data set is restricted to the constraining area.

11. The method of claim 1, wherein the identifying of the first image points is based on machine learning.

12. The method of claim 1, wherein the identifying of the first image points is based on the time-intensity curves of image points within a neighboring region of the respective image point.

13. A medical imaging device comprising:
a processor configured to:
capture an image data set of an examination object by a medical imaging device, wherein the image data set has a plurality of image points in each case with a time-intensity curve, and wherein the plurality of image points maps an examination area of the examination object with at least one contrast-enhanced vascular section;
identify first image points of the plurality of image points in the image data set comprising time-intensity curves that have a predefined variability as image points that map the at least one contrast-enhanced vascular section; and
provide the classified data set based on the image data set and the first image points,
wherein the classified data set has a classification between the first image points and further image points of the image data set.

14. The medical imaging device of claim 13, wherein the medical imaging device is a medical X-ray device, and
wherein the medical X-ray device is configured to capture projection images of the examination object.

15. A computer program product with a computer program configured to be loaded directly into a memory of a processor, wherein the computer program, when executed by the processor, is configured to:
capture an image data set of an examination object by a medical imaging device, wherein the image data set has a plurality of image points in each case with a time-intensity curve, and wherein the plurality of image points maps an examination area of the examination object with at least one contrast-enhanced vascular section;
identify first image points of the plurality of image points in the image data set comprising time-intensity curves that have a predefined variability as image points that map the at least one contrast-enhanced vascular section; and
provide the classified data set based on the image data set and the first image points,
wherein the classified data set has a classification between the first image points and further image points of the image data set.
